(19) United States
(12) Patent Application Publication
(10) Pub. No.: US 2009/0016075 A1
(43) Pub. Date: Jan. 15, 2009

(54) SEMICONDUCTOR LIGHTING IN CONSOLE SYSTEM FOR ILLUMINATING BIOLOGICAL TISSUES

(75) Inventors: Prashant Bhadri, Pico Rivera, CA (US); Ralph Kerns, Laguna Niguel, CA (US); Mark S. Humayun, Glendale, CA (US); Charles DeBoer, Pasadena, CA (US); Matthew McCormick, Forest Falls, CA (US); Sophia Fang, Los Angeles, CA (US)

Correspondence Address: MCDERMOTT WILL & EMERY LLP, 28 STATE STREET, BOSTON, MA, 02109-1775, US

(73) Assignee: Doheny Eye Institute, Los Angeles, CA (US)

(21) Appl. No.: 12/172,704

(22) Filed: Jul. 14, 2008

Related U.S. Application Data
(60) Provisional application No. 60/959,236, filed on Jul. 12, 2007.

(51) Int. Cl. F21V 7/04; A61B 1/06

(52) U.S. Cl. 362/555; 362/572

(57) ABSTRACT

The present disclosure is directed to illumination techniques that include the use of one or more sets of Light Emitting Diodes or LEDs as light sources in a console/module system. The LED light sources can be utilized to produce a light beams with a specified/combination of intensity and spectrum. Of course, embodiments according to the present disclosure are not limited to one intensity/spectrum but multiple combinations of intensity and spectrum can be implemented. Such systems/methods can be implemented with various optical elements including filter, lenses, mirrors, and/or optical fibers. The system is controlled by voice activation, touch screen, footswitch or wireless communication. The LEDs might also be pulsed as a driving system. The optical fiber cable is tethered to the control.

### BACKGROUND

The concept of using bandwidth limited wavelength light to enhance visualization of the ocular fundus has been researched previously For example, the term “rotfreiem licht” or red-free light was introduced in 1925 to the ophthalmic community as a method that would enhance the visual contrast of anatomical details of the fundus. Red-free light is still clinically used today in fundus photography and examination of the nerve fiber layer.

In general terms, typical illumination methods employed during ophthalmic surgery consist of using either a tungsten halogen, metal halide, or xenon arc light source coupled into a fiber optic light guide approximately three meters in length. These sources have some illumination shortcomings for biological tissues.

Short arc lamps operate at elevated temperatures of up to 900°C. and contain very high pressure usually in excess of 10 atmospheres. Power consumption of a 1000-watt xenon arc light source can be very high which also corresponds to short bulb life.

### SUMMARY

The present disclosure provides systems, methods, techniques, and apparatus useful for illumination for ophthalmic procedures and/or other procedures (e.g., surgical) on other biological tissues. According to exemplary embodiments of the present disclosure, ophthalmic illumination systems are provided that include one or more sets of Light Emitting Diodes (LED) light sources in a console or in a separate module. For example, the LED light sources can be utilized to produce a light beams with a specified/combination of intensity and spectrum. Of course, embodiments according to the present disclosure are not limited to one intensity/spectrum but multiple combinations of intensity and spectrum can be implemented.

Further embodiments of techniques (e.g., systems and/or methods) according to the present disclosure can be, for example and without limiting the scope of the present disclosure, implemented with one or more of the following in any combination:

A reflector system used to collect the light of wide angle of divergence;

A lens system used to collect the light from single/multiple LEDs;

A filter device/apparatus/means used to generate a bandwidth limited wavelength light or a combination

The light beam with the bandwidth-limited/combinational spectral distribution may be caused to converge into a spot to be coupled in a collecting device, for example, fiber optic cable or the like.

The system can be controlled by voice activation, touch screen, footswitch or wireless communication. The optical fiber cable is tethered to the console. The LED's can also be pulsed from the electronic driver system, in exemplary embodiments.

Other features and advantages of the present disclosure will be understood upon reading and understanding the detailed description of exemplary embodiments, described herein, in conjunction with reference to the drawings.

### BRIEF DESCRIPTION OF DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIGS. 1A-1C depict LED optical source configurations, in accordance with exemplary embodiments of the present disclosure; this representation can include either LED's on a different substrate or multiple LED's on a single die

FIG. 2 depicts a diagrammatic view of a system according to an embodiment of the present disclosure; and

FIG. 3 depicts a block diagram representing a method according to an exemplary embodiment of the present disclosure.

While certain embodiments depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

Biological tissue illumination systems/methods/techniques according to the present disclosure include the use of Light Emitting Diodes also known as LED's in a module/console. Illumination systems can include one or more LED sources and delivery optics, e.g., lens, reflectors and optical fiber. Illumination systems/methods/techniques according to the present disclosure can allow improved visualization for medical techniques such as ophthalmic operations. For example, the use of bandwidth-limited/specific spectrally distributed (combination spectrum, with or without white light) wavelength light can allow physicians to operate with improved contrasts for visualization of specific structures in the eye.

As described previously, systems according to the present disclosure can include the LED, which are devices that convert electrical energy into optical energy. An LED is a semiconductor based device that emits an INCOHERENT NARROW SPECTRUM of light when stimulated electrically. This phenomenon is termed electroluminescence where the color (UV, Visible, IR) of light depends on the type of semiconductor material, e.g., the related bandgap properties of the semiconductor material(s) used in the LED(s). In exemplary embodiments, this can be either on a single substrate or multiple substrates.

As they are based on electronic component design, LED's are largely if not entirely immune from system vibrations. If the LED's are protected from dirt and moisture, the lifetime increases to thousands of hours, this is much higher than a normal light sources. LED based light sources operate at lower temperatures, and therefore dissipate low heat, thereby eliminating complex heat sink systems. Cost of a single LED system is exponentially less expensive than a stand light source system because of the simple packaging and lower power requirements. LED's available in multiple colors and have high output efficiency

There are also benefits of using bandwidth-limited wavelength/spectrally distributed (combination spectrum, with or without white light) illumination, as provided by techniques according to the present disclosure, to improve the contrast of fundus details and eliminate the loss of quality of image associated with chromatic aberrations. The visualization and documentation of fundus structures is much improved and details are distinguished that are invisible with white light. This technique improves the ability to differentiate fundus details because one can observe changes in their appearance under different wavelengths. In addition, the bandwidth-limited wavelength technique allows more accurate localization of structures with regard to depth in the stratified layers of the fundus.

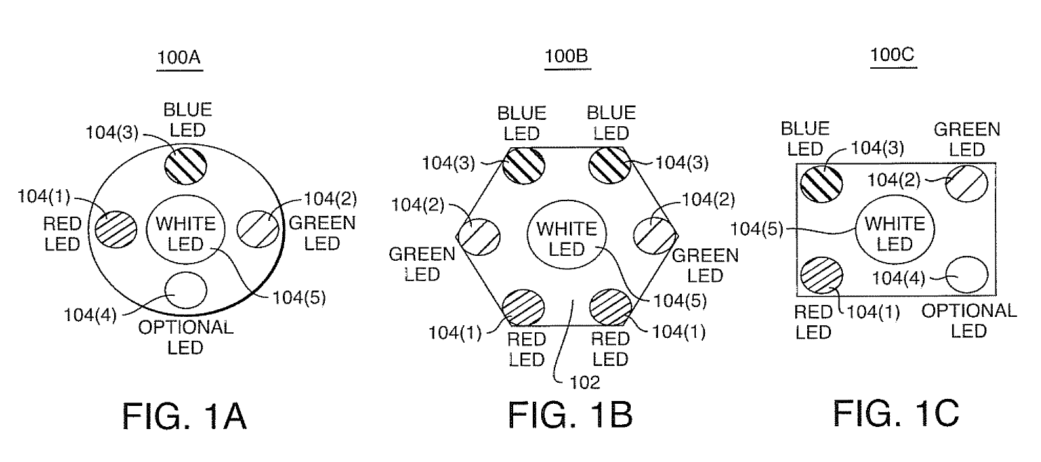

FIG. 1 includes FIGS. 1A-1C showing diagrammatic views representing exemplary embodiment's 100A-100C of components of biological tissue illumination systems according to the present disclosure. This representation can be either LED's on a different substrate or multiple LED's on a single die. For embodiments 100A-100C, a platform 102 (e.g., an assembly within a housing) can include multiple LEDs, as shown. The LEDs can be configured and operable so each produce light of a desired color (spectral range or bandwidth) and intensity or flux (e.g., Watts/cm2 or Watt/cm2/steradian, respectively), and can include one or more band width wavelengths (example: red) (LEDs 104(1), green LEDs, blue LED(s) 104(3), optional LEDs 104(4), and white LEDs 104(5), configured as light sources for surgical/diagnostics procedures, e.g., ophthalmic procedures in exemplary embodiments. This LED housing can be incorporated in a console/module which is a part of a system, or an independent by itself. While, exemplary configurations are illustrated in FIGS. 1A-1C, of course, other color LEDs may be used and the colors are not limited to the colors shown in the drawings. Also, any desired number of LEDs can be used, e.g., one or more as desired for a given application.

For example, in an exemplary embodiment, the LED light sources can be utilized for a surgical/diagnostics (e.g., ophthalmic) procedure to produce light beams with a specified/combination intensity and spectrum. The number/configuration of light sources (e.g., LEDs) is not limited, but multiple combinations of spectral distribution can be implemented.

In exemplary embodiments, a reflector system is used to collect the radiant energy into a column of light. A suitable coupling system can include a lens (e.g., lens system) and/or a filter (e.g., filter system) in suitable configurations that would allow physicians to select the level of intensity of the light used for illumination during a surgical/diagnostics procedure, e.g., an ophthalmic procedure. A lens system can be used to collect the light from multiple LED's. A filter device can be used to generate a bandwidth-limited wavelength of light. The light beam with the bandwidth-limited wavelength converges into a spot to be coupled in a collecting device, for example, fiber optic cable. Fiber Output Stage might consists of specialty fibers for selective outputs. The fiber can also be used as a filter by using thin film deposited fibers. The fiber optic can be tethered to the console. The LED light source(s) can be either controlled by various suitable control means, e.g., through features on a hand piece/handle or a surgical/diagnostics console. The system can be controlled by voice activation, touch screen, footswitch or wireless communication. The LED's can, in exemplary embodiments, be pulsed as a driving system. Communication is with a feedback loop to control the output of the light, the controls (controller and/or control means) can be located as desired. In exemplary embodiments, the controller is either in the handle or in the console and/or voice controlled.

With continued reference to FIG. 1, the selected LED light source(s) can provide the optical signal in a band of specific wavelengths. The different sources of different wavelengths (e.g., red, green, blue, white, etc.) can be configured in a designated pattern for maximum light output efficiency. The colors of any LED are not limited to the colors shown. Also, the number of LEDs can be multiple, and/or multiple colors can be implemented within/produced by one LED.

One of the advantages using this configuration is that by controlling the current to the LED, the Output light can be tuned at various intensities, which in turn can directly affect the spectrum, this allows for better safety/visualization tunable to individual cases and surgeons. In addition, the variation in light of different spectrum would allow for improved contrast ratios.

Embodiments of systems/methods according to the present disclosure can be used inside or outside (e.g., office based procedures) an operating room by providing improved structural viewing by color contrasts improvement during surgical/diagnostics procedures. Embodiments can provide improved contrast ratio by wavelength selection, spectral selectivity and/or wavelength filtering option and can provide variable color combination(s).

The intensity of the LED light can be controlled with a simple control system. In addition, LED light sources used can have a very long life, e.g., 50,000 hours or more. Systems according to the present disclosure can be immune from system vibrations, have an increased lifetime, low heat dissipation, simple packaging and multiple color output.

Further, embodiments can provide improved LED light output control during air/fluid exchange, greatly reduced energy costs, lower maintenance costs, fewer emissions of greenhouse gases, and/or reduced liability exposure.

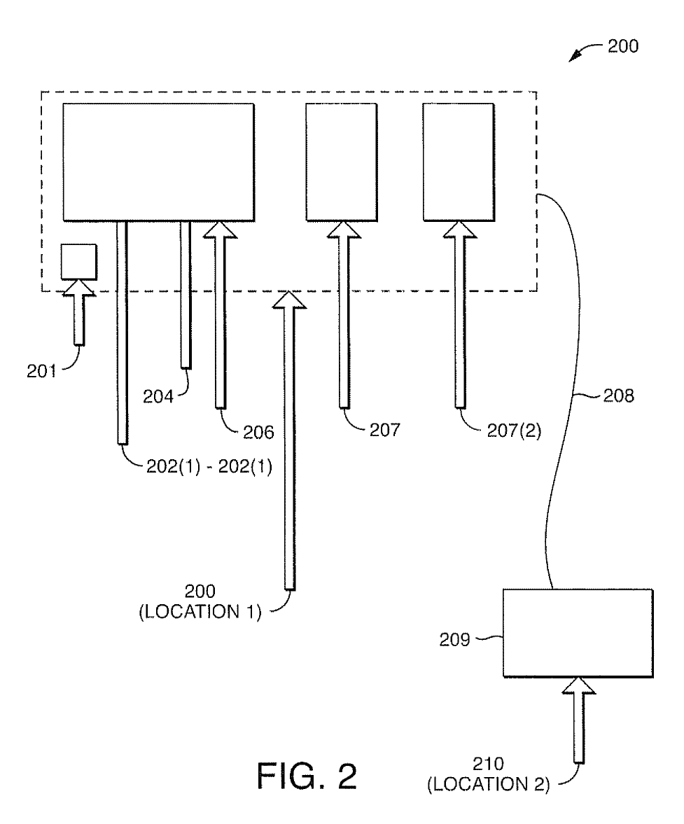

FIG. 2 depicts a diagrammatic view of a system 200 according to an embodiment of the present disclosure in a console. System 200 can include one or more LED light sources 202(1)-202(N) having desired output spectral characteristics and intensity(ies). The light source(s) 202(1)-202(N) can be located in a housing 204 or other suitable location. A collecting system/component/device 206 may be present to collect the optical output from the LED light source(s) 202(1)-202(N) and direct it. A converging component 207 consisting of lens filters helps in distinguishing the desired bandwidth spectrum. This couple relays the light into a optical fiber 208 or similar output component 208 can include a distal end 209 suitable to direct optical output to a biological tissues site for illumination.

As shown in FIG. 2, the collecting device 207 can include multiple components/parts, e.g., one or more optical fibers 208 (or other waveguide) and/or a 207 (lens) and/or reflector system 206. In exemplary embodiments, because of packaging limitations, the light from the one or more LED sources is preferably delivered from the LED(s)—also referred to herein as a peripheral illumination source—to the inside of the eye by means of fiber optic with resultant light loss.

With continued reference to FIG. 2, a power supply/source 201 may be present and configured to supply the light source(s) 202(1)-202(N) with sufficient power, such as for illumination of a biological tissues site during a surgical/diagnostics procedure. Power supply 201 may be located in housing 200 in exemplary embodiments. Control means for controlling the light output production (including possibly the spectral characteristics or colors) and/or intensity/fluence of the output can be located in/with system 200. This can include and not limited to pulsing the LED's through suitable drivers. The control means (or controller or control systems) 201 can include necessary or desired control electronics/circuitry and/or buttons, dials, and/or other adjustment components sufficient to allow a surgeon to selectively adjust and control the optical output from the one or more LED light sources during surgical/diagnostics procedure such as an ophthalmic procedure. Such control can include adjusting the contrast of the light incident upon and/or reflected from the biological tissues site.

In exemplary embodiments, the control means can be or include a user interface configured and arranged to control lighting for a surgical/diagnostics procedure. The user interface (and/or control means) can be controlled by the user by voice activation, touch screen, footswitch, electro/mechanical switch (e.g., in the handle of a surgical scope or probe) or via wireless communication. In exemplary embodiments, the one or more optical fibers can be arranged in an optical fiber cable that is tethered to a surgical console. The control means can be disposed in the handle of a surgical probe such as an endoscope, etc.

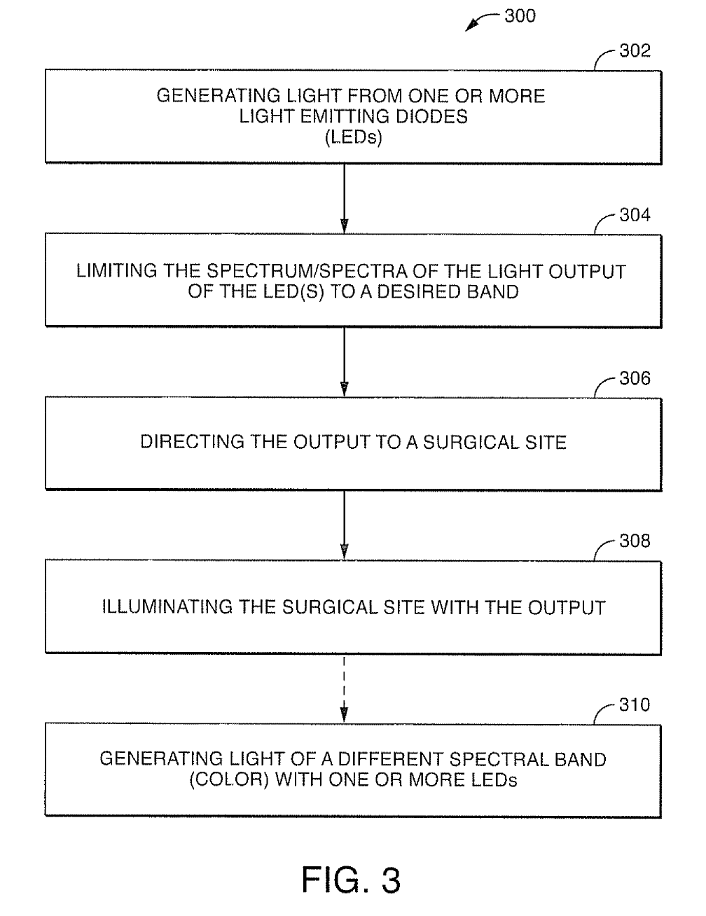

FIG. 3 depicts a block diagram representing a method 300 according to an exemplary embodiment of the present disclosure. According to the method 300, light can be generated from one or more LEDs either on different or one substrate, as described at 302 in a console/module. For example, light of desired red, green, and/or blue spectra can be generated by suitable LEDs. The light output of the LED(s) can be limited (filtered) spectrally to a desired bandwidth, as described at 304, such as used by an optical filtering element. This also means that the output spectral light can be controlled with or without a filtering element.

Continuing with the description of method 300, light from the LED(s) can be directed to a biological tissues site using optical fiber cable, as described at 306, for illuminating the site, as described at 308. Additional spectrums of light may likewise be generated and directed to the biological tissues site, in exemplary applications.

The light applied to a surgical/diagnostics procedure cab be controlled by use of a user interface. For example, the user interface can be controlled by voice activation, a touch screen, a footswitch, an electro/mechanical switch (such as on a handle of a surgical probe, e.g., endoscope probe), or wireless communication system/device. The light that is generated may be pulsed light, continuous light, or both.

### Exemplary Embodiments—Illuminated Biological Tissues System

LED based systems according to the present disclosure can offer significant advantages. The embodiments can provide one or more of the following benefits/advantages:

Improved Contrast ratio by wavelength selection;

Pulsing the input signal will allow spectral selectivity in the LED;

Allow Wavelength Filtering Option, as only selective LED can be turned;

Improved Light Control during the air/fluid interface by using;

Alternate potential applications for light sources according to the present disclosure can be for other illuminated instrument applications besides vitrectomy intraocular illumination: such as for application in an indirect ophthalmoscope, application in a direct ophthalmoscope, application in a slit lamp, application in a fundus camera, and others.

While certain embodiments have been described herein, it will be understood by one skilled in the art that the methods, systems, and apparatus of the present disclosure may be embodied in other specific forms without departing from the spirit thereof. For example, while certain geometric shapes have been shown and described specifically for exemplary embodiments of LED arrays, others may be used within the scope of the present disclosure.

Accordingly, the embodiments described herein are to be considered in all respects as illustrative of the present disclosure and not restrictive.

What is claimed:

1. An illumination system for surgical/diagnostics procedures, the system comprising:
one or more LEDs configured and arranged to emit white light responsive to power received from a power source;
control means for controlling intensity of white light emitted by the one or more LEDs; and
a collecting device configured and arranged to receive light from the one or more LEDs and direct the light to the biological tissues site.

2. The system of claim 1, further comprising one or more color tinted LEDs configured and arranged to produce light of desired spectral characteristics including bandwidth and/or intensity.

3. The system of claim 1, wherein the collecting device comprises one or more optical fibers.

4. The system of claim 1, wherein the collecting device comprises a reflector system, wherein the reflector system is configured and arranged to collect light of wide divergence.

5. The system of claim 1, wherein the converging device comprises a lens system used to collect the light from the one or more LEDs.

6. The system of claim 1, further comprising a filter device configured and arranged to receive light from the one or more LEDs and transmit a selective bandwidth limited/shifted wavelength light of a desired spectrum.

7. The system of claim 4, wherein the lens and filter device is configured and arranged to transmit a light beam of bandwidth limited wavelength that converges into a spot to be coupled into the collecting device.

8. The system of claim 1, wherein the control means comprise a user interface configured and arranged to control lighting for a surgical/diagnostics procedure.

9. The system of claim 8, wherein the user interface is controlled by the user by voice activation, touch screen, footswitch, electro/mechanical switch in the handle or wireless communication, and the optical fiber cable is tethered to the console.

10. The system of claim 8, wherein the control means are disposed in the handle of a surgical probe.

11. The system of claim 8, wherein the control means are disposed in a wireless communication system including a receiver and a transmitter.

12. The system of claim 3, wherein the one or more optical fibers are arranged in a cable tethered to a console.

13. The system of claim 1, wherein the one or more LEDs are configured and arranged to be pulsed from an electronic driving system.

14. The system of claim 1, wherein the one or more LEDs are configured and arranged for continuous operation by the control of an electronic driver system.

15. An illumination system for surgical/diagnostics procedures comprising:
one or more LEDs configured and arranged to emit single or/and a combination bandwidth wavelength light in response to power received from a power source;
a control device for controlling intensity and spectrum of individual or/and combination of bandwidth wavelength light emitted by the one or more LEDs; and
a collecting device configured and arranged to receive light from the one or more LEDs and direct the light to a biological tissue site.

16. The system of claim 15, wherein the one or more LEDs comprise a plurality of LEDs disposed on a single substrate or multiple different substrates.

17. The system of claim 15, wherein the collecting device is configured and arranged to transmit the light received from the one or more LEDs such that the transmitted light converges into a spot to be coupled into the collecting device.

18. The system of claim 16, wherein the one or more LEDs are directly coupled is to an optical fiber.

19. The system of claim 17, wherein the collecting device comprises a reflector system.

20. The system of claim 17, wherein the coupling device comprises a lens system configured and arranged to collect light from one or more LEDs.

21. The system of claim 15, further comprising a filter device configured and arranged to generate a bandwidth limited wavelength light in addition to the predefined bandwidth of the light of the one or more LEDs.

22. The system of claim 15, wherein the system is controlled by a user interface

23. The system of claim 22, wherein the user interface is controlled by voice activation, touch screen, footswitch, an electro/mechanical switch, or wireless communication.

24. The system of claim 18, wherein the optical fiber cable is tethered to a console.

25. A method of producing bandwidth limited light for surgical/diagnostics procedures comprising:
generating light of desired non-white spectra from one or more light emitting diodes;
limiting the spectra to a desired band;
directing the output to a biological tissues site; and
illuminating the biological tissues site with the output for a surgical/diagnostics procedure.

26. The method of claim 25, further comprising generating white light.

27. The method of claim 26, wherein the white light is directed to the biological tissues site.

28. The method of claim 25, further comprising controlling the light applied to a surgical/diagnostics procedure by use of a user interface.

29. The method of claim 28, wherein the user interface is controlled by voice activation, touch screen, footswitch, electro/mechanical switch, or wireless communication.

30. The method of claim 25, wherein generating light from one or more light emitting diodes comprises generating pulsed light, continuous light, or both.
